(19) United States
(12) Patent Application Publication
(10) Pub. No.: US 2005/0272957 A1
(43) Pub. Date: Dec. 8, 2005

(54) Process for preparing dibasic salts of bisphenols

(75) Inventors: Changlu Gao, Jilin Province (CN); Lianxun Gao, Jilin Province (CN); Mengxian Ding, Jilin Province (CN)

Correspondence Address: FISH & RICHARDSON PC, P.O. BOX 1022, MINNEAPOLIS, MN, 55440-1022, US

(73) Assignee: CHANGCHUN INSTITUTE OF APPLIED CHEMISTRY

(21) Appl. No.: 11/120,563

(22) Filed: May 3, 2005

(30) Foreign Application Priority Data
Jun. 3, 2004 (CN) 2004100109000

(51) Int. Cl. C07C 39/12; C07C 49/786

(52) U.S. Cl. 568/53; 568/718; 568/722; 568/337; 568/662

(57) ABSTRACT

The present invention discloses a process for preparing dibasic salt of bisphenol from bisphenol and alkali, wherein the bisphenol and the alkali are reacted in a reaction medium consisting of an organic solvent or an aqueous inorganic salt solution, and then through filtration, a highly pure dibasic salt of bisphenol is directly obtained at a yield of higher than 90%.

### FIELD OF TECHNOLOGY

The present invention relates to a process for preparing dibasic salts of bisphenols, in particular, to a process for preparing dibasic salts of bisphenols by reacting a bisphenol compound and an alkali in a reaction medium consisting of an organic solvent or an aqueous inorganic salt solution.

### BACKGROUND ART

Poly(aryl ether sulfone), poly(ether ketone) and polyetherimide are the most widely used heat-resisting polymeric materials nowadays. Among them, great attention is especially paid to polyetherimide due to its outstanding properties and potential wide applications. Poly(aryl ether ketone) and poly(aryl ether sulfone) are conventionally prepared by polymerizing a bisphenol monomer with a bis-halogen monomer in the presence of an excess alkali in aprotic polar solvent such as N-methylpyrrolidone through azeotropic dewatering. However, azeotropic dewatering could not assure the complete conversion of bisphenols and the product thus obtained often contains unconverted monobasic salt. As a result, the yield of dibasic salts of bisphenols is low, the repeatability of the polymerization is poor and it is difficult to obtain high molecular weight polymers. The better heat-resistant material, polyetherimide, is at present often prepared by first synthesizing bisether-bisanhydride and then polymerizing the bisether-bisanhydride with a diamine. The process of directly polymerizing a disubstituted bis-phthalimide monomer with a bisphenol is considered as the most economical one for preparing polyetherimide, yet this process has not been industrialized. The one of the important reason for this lies in the difficulty of obtaining highly pure dibasic salts of bisphenols in high yield. In 1997, GE Company disclosed a process for preparing disodium salts of bisphenols (U.S. Pat. No. 5,663,275), but the process had a yield of merely 50%. Thus there is a need for a novel process by which dibasic salts of bisphenols could be prepared in high yield and in high purity.

### DISCLOSURES OF THE INVENTION

The objective of the present invention is to provide a process for preparing dibasic salts of bisphenols from bisphenols and alkalis.

A bisphenol and an alkali are reacted in a reaction medium consisting of an organic solvent or an aqueous inorganic salt solution, and then through filtration, a highly pure dibasic salt of bisphenol is directly obtained at a yield of higher than 90%.

Examples of suitable bisphenols useful in the process according to the present invention include, but are not limited to:

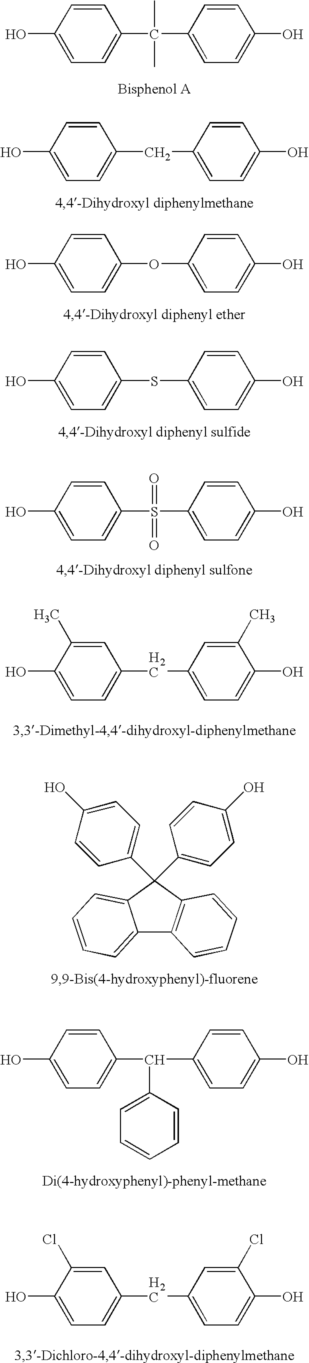

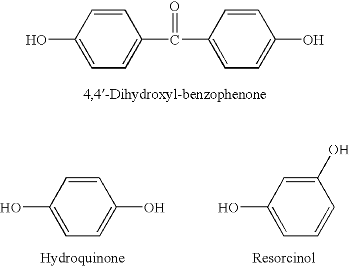

Examples of suitable alkalis useful in the process according to the present invention include, but are not limited to, potassium hydroxide, sodium hydroxide, sodium carbonate and potassium carbonate.

Examples of the reaction medium useful in the process according to the present invention include, but are not limited to, acetone, butanone, cyclohexanone, ethanol, isopropyl alcohol, n-propyl alcohol, n-butyl alcohol, isobutyl alcohol, dioxane, tetrahydrofuran, 10-28 wt. % aqueous sodium chloride solution, 6-14 wt. % aqueous sodium bicarbonate solution, 10-37wt. % aqueous potassium chloride solution, 7-32 wt. % aqueous sodium carbonate solution, 10-37 wt. % aqueous potassium bicarbonate solution or 10-60 wt. % aqueous potassium carbonate solution.

In the process of the present invention, in order to assure the completion of the reaction and to simplify the post-treatment, the molar ratio of the bisphenol and the alkali may be controlled in a range of from 1:2 to 1:2.05, and the amount of the reaction medium used should be in a range of from 0.6 to 2.0 liter per mole of the bisphenol.

The reaction is preferably carried out in the absence of oxygen, such as under nitrogen atmosphere. The reaction temperature could be in a range of from 20 to 100° C., and the reaction time may vary from 1 to 5 hours.

The product of the reaction, dibasic salt of bisphenol, could be obtained in the form of precipitate, and post-treated with conventional methods such as conventional washing and drying. In a preferred embodiment, the resultant product was first washed with hot solvent or cool water as described above, and then dried under reduced pressure at a temperature of from 200 to 250° C., to yield anhydrous dibasic salt of bisphenol. The yield of the process according to the present invention could attain 90 to 95%.

Using the process of the present invention, highly pure dibasic salts of bisphenols could be conveniently obtained at high yield.

Embodiments Of The Invention

The following examples further describe the invention, but do not make limitation to the scope of the invention in any way.

### EXAMPLE 1

0.01 Mole of bisphenol A, 0.02 mole of NaOH and 200 mL of isopropyl alcohol were charged into a 250 mL three necked flask, and nitrogen gas was bubbled to remove oxygen in the flask. The mixture was allowed to react at 60° C. for 1 hr, and a precipitate was obtained. The precipitate was filtered out under nitrogen atmosphere, washed twice with 30 mL of hot isopropyl alcohol, and then dried under reduced pressure at 200° C. for 3 hrs, to yield 25.6 g of bis-sodium salt of bisphenol A. The yield was 94%, and the purity was measured by titration to be 99.9%.

### EXAMPLE 2

0.01 Mole of bisphenol A, 0.02 mole of NaOH and 200 mL of n-propyl alcohol were charged into a 250 mL three necked flask, and nitrogen gas was bubbled to remove oxygen in the flask. The mixture was allowed to react at 60° C. for 3 hrs, and a precipitate was obtained. The precipitate was filtered out under nitrogen atmosphere, washed twice with 30 mL of hot n-propyl alcohol, and then dried under reduced pressure at 200° C. for 3 hrs, to yield 25.0 g of bis-sodium salt of bisphenol A. The yield was 92%, and the purity was measured by titration to be 99.9%.

### EXAMPLE 3

0.01 Mole of bisphenol A, 0.02 mole of NaOH and 200 mL of isobutyl alcohol were charged into a 250 mL three necked flask, and nitrogen gas was bubbled to remove oxygen in the flask. The mixture was allowed to react at 60° C. for 1 hr, and a precipitate was obtained. The precipitate was filtered out under nitrogen atmosphere, washed twice with 30 mL of hot isobutyl alcohol, and then dried under reduced pressure at 200° C. for 3 hrs, to yield 25.6 g of bis-sodium salt of bisphenol A. The yield was 94%, and the purity was measured by titration to be 99.9%.

### EXAMPLE 4

0.01 Mole of 4,4′-dihydroxyl diphenylmethane, 0.02 mole of KOH and 150 mL of butanone were charged into a 250 mL three necked flask, and nitrogen gas was bubbled to remove oxygen in the flask. The mixture was allowed to react at 80° C. for 5 hrs, and a precipitate was obtained. The precipitate was filtered out under nitrogen atmosphere, washed twice with 50 mL of hot butanone, and then dried under reduced pressure at 220° C. for 5 hrs, to yield 26.2 g of bis-potassium salt of 4,4′-dihydroxyl diphenylmethane. The yield was 95%, and the purity was measured by titration to be 99.9%.

### EXAMPLE 5

0.01 Mole of 4,4′-dihydroxyl diphenyl ether, 0.02 mole of NaOH and 200 mL of butanone were charged into a 250 mL three necked flask, and nitrogen gas was bubbled to remove oxygen in the flask. The mixture was allowed to react at 100° C. for 3 hrs, and a precipitate was obtained. The precipitate was filtered out under nitrogen atmosphere, washed twice with 50 mL of hot butanone, and then dried under reduced pressure at 200° C. for 4 hrs, to yield 23.3 g of bis-sodium salt of 4,4′-dihydroxyl diphenyl ether. The yield was 95%, and the purity was 99.7%.

### EXAMPLE 6

0.01 Mole of 4,4′-dihydroxyl diphenyl thioether, 0.021 mole of NaOH and 100 mL of cyclohexanone were charged into a 250 mL three necked flask, and nitrogen gas was bubbled to remove oxygen in the flask. The mixture was allowed to react at 100° C. for 4 hrs, and a precipitate was obtained. The precipitate was filtered out under nitrogen atmosphere, washed twice with 50 mL of hot cyclohexanone, and then dried under reduced pressure at 200° C. for 5 hrs, to yield 24.6 g of bis-sodium salt of 4,4′-dihydroxyl diphenyl thioether. The yield was 94%, and the purity was 99.8%.

### EXAMPLE 7

0.01 Mole of 4,4′-dihydroxyl diphenyl sulfone, 0.02 mole of NaOH and 100 mL of cyclohexanone were charged into a 250 mL three necked flask, and nitrogen gas was bubbled to remove oxygen in the flask. The mixture was allowed to react at 60° C. for 4 hrs, and a precipitate was obtained. The precipitate was filtered out under nitrogen atmosphere, washed twice with 50 mL of hot cyclohexanone, and then dried under reduced pressure at 240° C. for 5 hrs, to yield 27.5 g of bis-potassium salt of 4,4′-dihydroxyl diphenyl sulfone. The yield was 93%, and the purity was 99.9%.

### EXAMPLE 8

0.01 Mole of 3,3′-dimethyl-4,4′-dihydroxyl diphenylmethane, 0.02 mole of NaOH and 60 mL of n-butyl alcohol were charged into a 250 mL three necked flask, and nitrogen gas was bubbled to remove oxygen in the flask. The mixture was allowed to react at 60° C. for 4 hrs, and a precipitate was obtained. The precipitate was filtered out under nitrogen atmosphere, washed twice with 50 mL of hot n-butyl alcohol, and then dried under reduced pressure at 250° C. for 3 hrs, to yield 25.8 g of bis-sodium salt of 3,3′-dimethyl-4,4′-dihydroxyl diphenylmethane. The yield was 95%, and the purity was 99.9%.

### EXAMPLE 9

0.01 Mole of 9,9-bis(4-hydroxyphenyl)fluorene, 0.02 mole of NaOH and 100 mL of acetone were charged into a 250 mL three necked flask, and nitrogen gas was bubbled to remove oxygen in the flask. The mixture was allowed to react at 50° C. for 4 hrs, and a precipitate was obtained. The precipitate was filtered out under nitrogen atmosphere, washed twice with 50 mL of hot acetone, and then dried under reduced pressure at 250° C. for 3 hrs, to yield 37 g of bis-sodium salt of 9,9-bis(4-hydroxyphenyl)fluorene. The yield was 94%, and the purity was 99.8%.

### EXAMPLE 10

0.01 Mole of di(4-hydroxyphenyl)-phenyl-methane, 0.02 mole of NaOH and 100 mL of acetone were charged into a 250 mL three necked flask, and nitrogen gas was bubbled to remove oxygen in the flask. The mixture was allowed to react at 60° C. for 4 hrs, and a precipitate was obtained. The precipitate was filtered out under nitrogen atmosphere, washed twice with 50 mL of hot acetone, and then dried under reduced pressure at 200° C. for 3 hrs, to yield 30 g of bis-sodium salt of di(4-hydroxyphenyl)-phenyl-methane. The yield was 94%, and the purity was 99.9%.

### EXAMPLE 11

0.01 Mole of 3,3′-dichloro-4,4′-dihydroxyl-diphenyl methane, 0.02 mole of NaOH and 120 mL of acetone were charged into a 250 mL three necked flask, and nitrogen gas was bubbled to remove oxygen in the flask. The mixture was refluxed for 4 hrs, and a precipitate was obtained. The precipitate was filtered out under nitrogen atmosphere, washed twice with 50 mL of hot acetone, and then dried under reduced pressure at 200° C. for 3 hrs, to yield 29.4 g of bis-sodium salt of 3,3′-dichloro-4,4′-dihydroxyl-diphenyl methane. The yield was 94%, and the purity was 99.7%.

### EXAMPLE 12

0.01 Mole of 4,4′-dihydroxyl-benzophenone, 0.02 mole of NaOH and 150 mL of acetone were charged into a 250 mL three necked flask, and nitrogen gas was bubbled to remove oxygen in the flask. The mixture was allowed to react at 20° C. for 4 hrs, and a precipitate was obtained. The precipitate was filtered out under nitrogen atmosphere, washed twice with 50 mL of hot acetone, and then dried under reduced pressure at 200° C. for 3 hrs, to yield 24.2 g of bis-sodium salt of 4,4′-dihydroxyl-benzophenone. The yield was 94%.

### EXAMPLE 13

0.01 Mole of bisphenol A, 0.0205 mole of NaOH and 100 mL of n-butyl alcohol were charged into a 250 mL three necked flask, and nitrogen gas was bubbled to remove oxygen in the flask. The mixture was allowed to react at 50° C. for 4 hrs, and a precipitate was obtained. The precipitate was filtered out under nitrogen atmosphere, washed twice with 50 mL of hot n-butyl alcohol, and then dried under reduced pressure at 200° C. for 3 hrs, to yield 25.6 g of bis-sodium salt of bisphenol A. The yield was 94%, and the purity was 99.9%.

### EXAMPLE 14

0.01 Mole of bisphenol A, 0.02 mole of NaOH and 200 mL of acetone were charged into a 250 mL three necked flask, and nitrogen gas was bubbled to remove oxygen in the flask. The mixture was allowed to react at 60° C. for 1 hr, and a precipitate was obtained. The precipitate was filtered out under nitrogen atmosphere, washed twice with 30 mL of hot acetone, and then dried under reduced pressure at 200° C. for 3 hrs, to yield 25.0 g of bis-sodium salt of bisphenol A. The yield was 92%, and the purity was 99.9%.

### EXAMPLE 15

0.01 Mole of 3,3′-dichloro-4,4′-dihydroxyl-diphenyl methane, 0.02 mole of NaOH and 120 mL of ethanol were charged into a 250 mL three necked flask, and nitrogen gas was bubbled to remove oxygen in the flask. The mixture was refluxed for 4 hrs, and a precipitate was obtained. The precipitate was filtered out under nitrogen atmosphere, washed twice with 50 mL of hot ethanol, and then dried under reduced pressure at 200° C. for 3 hrs, to yield 28.4 g of bis-sodium salt of 3,3′-dichloro-4,4′-dihydroxyl-diphenyl methane. The yield was 91%, and the purity was 99.9%.

### EXAMPLE 16

0.01 Mole of 3,3′-dichloro-4,4′-dihydroxyl-diphenyl methane, 0.02 mole of NaOH and 120 mL of dioxane were charged into a 250 mL three necked flask, and nitrogen gas was bubbled to remove oxygen in the flask. The mixture was refluxed for 4 hrs, and a precipitate was obtained. The precipitate was filtered out under nitrogen atmosphere, washed twice with 50 mL of hot dioxane, and then dried under reduced pressure at 200° C. for 3 hrs, to yield 28.4 g of bis-sodium salt of 3,3′-dichloro-4,4′-dihydroxyl-diphenyl methane. The yield was 92%, and the purity was 99.7%.

### EXAMPLE 17

0.01 Mole of 3,3′-dichloro-4,4′-dihydroxyl-diphenyl methane, 0.02 mole of NaOH and 120 mL of tetrahydrofuran were charged into a 250 mL three necked flask, and nitrogen gas was bubbled to remove oxygen in the flask. The mixture was refluxed for 4 hrs, and a precipitate was obtained. The precipitate was filtered out under nitrogen atmosphere, washed twice with 50 mL of cool water, and then dried under reduced pressure at 200° C. for 3 hrs, to yield 28.4 g of bis-sodium salt of 3,3′-dichloro-4,4′-dihydroxyl-diphenyl methane. The yield was 91%, and the purity was 99.7%.

### EXAMPLE 18

0.01 Mole of hydroquinone, 0.02 mole of NaOH and 100 mL of 26% aqueous NaCl solution were charged into a 250 mL three necked flask, and nitrogen gas was bubbled to remove oxygen in the flask. The mixture was refluxed for 4 hrs, and a precipitate was obtained. The precipitate was filtered out under nitrogen atmosphere, washed twice with 50 mL of cool water, and then dried under reduced pressure at 200° C. for 3 hrs, to yield 13.8 g of bis-sodium salt of hydroquinone. The yield was 90%, and the purity was 99.9%.

### EXAMPLE 19

0.01 Mole of resorcinol, 0.02 mole of NaOH and 100 mL of 10% aqueous NaCl solution were charged into a 250 mL three necked flask, and nitrogen gas was bubbled to remove oxygen in the flask. The mixture was refluxed for 4 hrs, and a precipitate was obtained. The precipitate was filtered out under nitrogen atmosphere, washed twice with 50 mL of hot 15% aqueous NaCl solution, and then dried under reduced pressure at 200° C. for 3 hrs, to yield 14.5 g of bis-sodium salt of resorcinol. The yield was 94%, and the purity was 99.8%.

### EXAMPLE 20

0.01 Mole of bisphenol A, 0.021 mole of Na₂CO₃ and 200 mL of 8.4% aqueous sodium bicarbonate solution were charged into a 250 mL three necked flask, and nitrogen gas was bubbled to remove oxygen in the flask. The mixture was allowed to react at 100° C. for 1 hr, and a precipitate was obtained. The precipitate was filtered out under nitrogen atmosphere, washed twice with 30 mL of hot isopropyl alcohol, and then dried under reduced pressure at 200° C. for 3 hrs, to yield 25.6 g of bis-sodium salt of bisphenol A. The yield was 94%, and the purity was 99.9%.

### EXAMPLE 21

0.01 Mole of bisphenol A, 0.021 mole of K₂CO₃ and 150 mL of 26% aqueous potassium chloride solution were charged into a 250 mL three necked flask, and nitrogen gas was bubbled to remove oxygen in the flask. The mixture was allowed to react at 100° C. for 1 hr, and a precipitate was obtained. The precipitate was filtered out under nitrogen atmosphere, washed twice with 30 mL of hot isopropyl alcohol, and then dried under reduced pressure at 200° C. for 3 hrs, to yield 28.6 g of bis-potassium salt of bisphenol A. The yield was 94%, and the purity was 99.8%.

### EXAMPLE 22

0.01 Mole of 4,4′-dihydroxyl diphenylmethane, 0.021 mole of K₂CO₃ and 150 mL of 10% aqueous potassium chloride solution were charged into a 250 mL three necked flask, and nitrogen gas was bubbled to remove oxygen in the flask. The mixture was allowed to react at 100° C. for 1 hr, and a precipitate was obtained. The precipitate was filtered out under nitrogen atmosphere, washed twice with 30 ML of hot isopropyl alcohol, and then dried under reduced pressure at 200° C. for 3 hrs, to yield 24.6 g of bis-potassium salt of bisphenol A. The yield was 90%, and the purity was 99.6%.

### EXAMPLE 23

0.01 Mole of bisphenol A, 0.02 mole of NaOH and 150 mL of 10% aqueous sodium carbonate solution were charged into a 250 mL three necked flask, and nitrogen gas was bubbled to remove oxygen in the flask. The mixture was allowed to react at 100° C. for 1 hr, and a precipitate was obtained. The precipitate was filtered out under nitrogen atmosphere, washed twice with 30 mL of hot isopropyl alcohol, and then dried under reduced pressure at 200° C. for 3 hrs, to yield 24.6 g of bis-sodium salt of bisphenol A. The yield was 90%, and the purity was 99.7%.

### EXAMPLE 24

0.01 Mole of bisphenol A, 0.02 mole of NaOH and 150 mL of 18% aqueous sodium carbonate solution were charged into a 250 mL three necked flask, and nitrogen gas was bubbled to remove oxygen in the flask. The mixture was allowed to react at 100° C. for 1 hr, and a precipitate was obtained. The precipitate was filtered out under nitrogen atmosphere, washed twice with 30 mL of hot isopropyl alcohol, and then dried under reduced pressure at 200° C. for 3 hrs, to yield 25.0 g of bis-sodium salt of bisphenol A. The yield was 93%, and the purity was 99.9%.

### EXAMPLE 25

0.01 Mole of bisphenol A, 0.02 mole of NaOH and 150 mL of 20% aqueous KHCO₃ solution were charged into a 250 mL three necked flask, and nitrogen gas was bubbled to remove oxygen in the flask. The mixture was allowed to react at 100° C. for 1 hr, and a precipitate was obtained. The precipitate was filtered out under nitrogen atmosphere, washed twice with 30 mL of hot isopropyl alcohol, and then dried under reduced pressure at 200° C. for 3 hrs, to yield 25.8 g of bis-sodium salt of bisphenol A. The yield was 95%.

### EXAMPLE 26 0.01 Mole of bisphenol A, 0.02 mole of NaOH and 150 mL of 10% aqueous K₂CO₃ solution were charged into a 250 mL three necked flask, and nitrogen gas was bubbled to remove oxygen in the flask. The mixture was allowed to react at 100° C. for 1 hr, and a precipitate was obtained. The precipitate was filtered out under nitrogen atmosphere, washed twice with 30 mL of hot isopropyl alcohol, and then dried under reduced pressure at 200° C. for 3 hrs, to yield 25.8 g of bis-sodium salt of bisphenol A. The yield was 95%.

### EXAMPLE 27

0.01 Mole of bisphenol A, 0.02 mole of NaOH and 150 mL of 35% aqueous K₂CO₃ solution were charged into a 250 mL three necked flask, and nitrogen gas was bubbled to remove oxygen in the flask. The mixture was allowed to react at 100° C. for 1 hr, and a precipitate was obtained. The precipitate was filtered out under nitrogen atmosphere, washed twice with 30 mL of hot isopropyl alcohol, and then dried under reduced pressure at 200° C. for 3 hrs, to yield 25.8 g of bis-sodium salt of bisphenol A. The yield was 95%.

1. A process for preparing dibasic salts of bisphenols, comprising reacting a bisphenol compound and an alkali under inert atmosphere in a reaction medium consisting of an organic solvent or an aqueous inorganic salt solution, to form a product in the form of precipitate.

2. The process for preparing dibasic salts of bisphenols according to claim 1, wherein the bisphenol is selected from the group consisting of

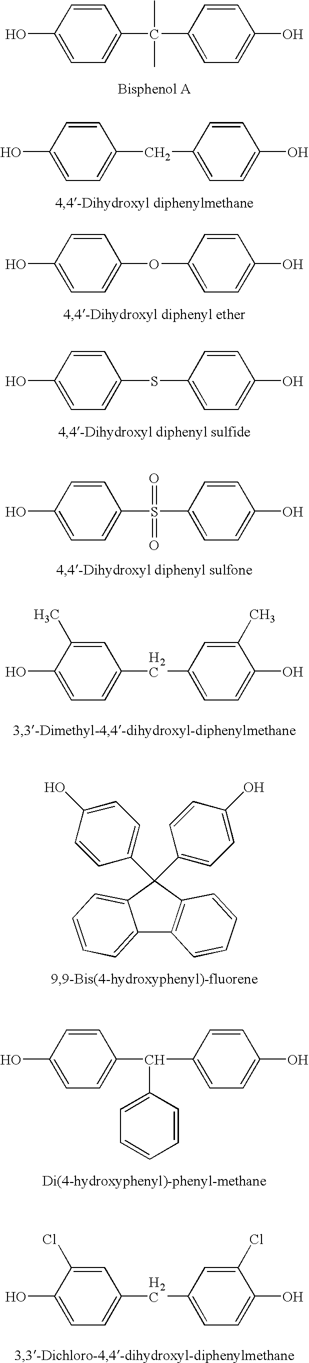

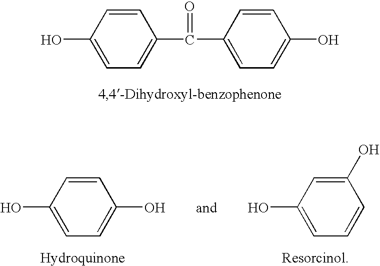

3. The process for preparing dibasic salts of bisphenols according to claim 1, wherein the alkali is selected from the group consisting of potassium hydroxide, sodium hydroxide, sodium carbonate and potassium carbonate.

4. The process for preparing dibasic salts of bisphenols according to claim 1, wherein the reaction medium is selected from the group consisting of acetone, butanone, cyclohexanone, ethanol, isopropyl alcohol, n-propyl alcohol, n-butyl alcohol, isobutyl alcohol, dioxane, tetrahydrofuran, 10-28 wt. % aqueous sodium chloride solution, 6-14 wt. % aqueous sodium bicarbonate solution, 10-37 wt. % aqueous potassium chloride solution, 7-32 wt. % aqueous sodium carbonate solution, 10-37 wt. % aqueous potassium bicarbonate solution and 10-60 wt. % aqueous potassium carbonate solution.

5. The process for preparing dibasic salts of bisphenols according to claim 1, wherein molar ratio of the bisphenol to the alkali is in a range of from 1:2 to 1:2.05.

6. The process for preparing dibasic salts of bisphenols according to claim 1, wherein amount of the reaction medium used varies from 0.6 to 2.0 L/mole of bisphenol.

7. The process for preparing dibasic salts of bisphenols according to claim 1, wherein the reaction is carried out under an oxygen-free atmosphere.

8. The process for preparing dibasic salts of bisphenols according to claim 1, wherein reaction temperature is in a range of from 20 to 100° C.

9. The process for preparing dibasic salts of bisphenols according to claim 1, wherein reaction time is in a range of from 1 to 5 hrs.

10. The process for preparing dibasic salts of bisphenols according to claim 1, further comprising filtering out the reaction product, washing it with a solvent and then drying the product under reduced pressure at a temperature of from 200 to 250° C. for 3 to 5 hrs.
